(19) United States
(12) Patent Application Publication
(10) Pub. No.: US 2011/0106033 A1
(43) Pub. Date: May 5, 2011

(54) OSTOMY APPLIANCES WITH SULFONATED POLYMERS

(75) Inventor: Mahesh Sambasivam, Pennington, NJ (US)

(73) Assignee: Conva Tec Technologies Inc.

(21) Appl. No.: 12/999,642

(22) PCT Filed: Jun. 19, 2009
(86) PCT No.: PCT/US09/47984

§ 371 Date: Dec. 17, 2010

Related U.S. Application Data
(60) Provisional application No. 61/073,979, filed on Jun. 19, 2008.

(51) Int. Cl. A61L 24/04

(52) U.S. Cl. 604/344

(57) ABSTRACT

This invention relates to an ostomy appliance comprising a body side component, a biocompatible pressure sensitive adhesive component for attaching the said bodyside component to the peristomal area and/or stoma, wherein the biocompatible pressure sensitive adhesive component includes at least one polymer with sulfonated functional group.

### BACKGROUND OF THE INVENTION

There are several medical conditions that require attachment of a device to the body such as ostomy, to collect waste matter. These devices are typically attached to the body via a pressure sensitive adhesive. An ostomy appliance or device typically includes a wafer containing a pressure sensitive adhesive and a receiving pouch or bag to collect and contain body waste. When the pouch is removably attached to the wafer via a coupling mechanism, it is referred to as a two-piece device. When the wafer and the pouch are permanently attached to each other, the device is referred to as a one-piece device. The ability of the wafer to adhere to the body is primarily determined by the pressure sensitive adhesive. Unlike other devices that are secured to the body via a pressure sensitive adhesive with minimal or no load on the adhesive, in an ostomy device or appliance, the adhesive has to not only securely hold the device against the body but also be able to bear the weight of the pouch when it gets filled with body waste.

Prior to application of the appliance, the user trims the opening of the adhesive portion of the one-piece or two-piece appliance to fit around the stoma. The pressure sensitive adhesives used in ostomy appliances are typically based on hydrocolloids. The hydrocolloids are mixed into a matrix of a pressure sensitive adhesive composition wherein the pressure sensitive adhesive composition provides adhesion to the skin under dry conditions, and the hydrocolloids provide adhesion to the skin under wet/moist conditions. A significant challenge in adhesion for an ostomy application is the maintenance of sufficient adhesion under wet conditions when the skin perspires and when the adhesive comes in contact with the stomal effluent. The adhesion provided by the hydrocolloids of the conventional ostomy adhesives is not sufficient to maintain a secure adhesion to the body under these conditions.

When the adhesive portion close to the stoma is exposed to fecal matter, the adhesion may be compromised, and the appliance can leak causing embarrassment to the user. If this happens, frequently, the peristomal skin health is also affected which may have a serious impact to the user. Hence, it is desirable to have an appliance that prevents leakage of intestinal effluent in the peristomal area by securely adhering to the body, and directing the effluent into the collection bag.

It is the objective of the present invention to provide a biocompatible pressure sensitive adhesive for securely adhering ostomy appliances to the body and/or to the stoma. In order to achieve this, the pressure sensitive adhesive should contain components that have specific interaction with the mucous membrane and also bond securely to the skin. Adhesives that adhere to mucous membrane are termed as mucoadhesives. Mucoadhesives are widely used in drug delivery to the buccal cavity or the gastrointestinal tract. However, such adhesives are either not pressure sensitive adhesives or they do not have the shear or wet strength to bear the load of an ostomy pouch. Prior to the present invention, pressure sensitive adhesives with mucoadhesive components have not been considered for use in an ostomy appliance to provide adhesion simultaneously to the body and to the stoma, and to maintain adhesion under dry and wet conditions.

The biocompatible adhesives of the present invention are based on polymers with functional groups that have specific interaction with the mucous membrane, preferably sulfonated functional groups.

Some patent documents related to the present invention are discussed below:

U.S Pat. No. 5,066,694 discloses pressure sensitive adhesive compositions with copolymers of isoprene and sodium styrene sulfonate that are prepared by emulsion polymerization to eliminate solvent. There is no mention of mucoadhesion in this invention.

U.S. Pat. No. 5,614,586 discloses hydrogel pressure sensitive adhesive compositions for attaching biomedical electrodes to skin formed by polymerizing a water soluble soluble long chain (meth)acrylate ester monomer with monomers containing sulfonate, acid, acrylamide or phosphonate groups. There is no mention of mucoadhesion in this invention.

U.S. Pat. No. 5,700,478 discloses water soluble pressure sensitive mucoadhesive that can attach to the mucous lining and to a variety of materials that constitute a device or prosthesis and for emplacement of such devices in a body cavity. The authors disclose a composition based on highly water soluble polymers and plasticizers that will leach away in the presence of water or moisture.

U.S. Pat. No. 7,138,458 B2 discloses the preparation of a two-phase, bioadhesive composition comprising an admixture of a crosslinkable hydrophobic composition and a hydrophilic composition. Since the hydrophilic polymer is blended into the composition, it can leach out in the presence of water or moisture.

WO20060927991 discloses a mucoadhesive polymer with a ligand attached to the polymer backbone to interact with toxins.

U.S. Patent Publication No. 2006/0054276 discloses a composition for attaching two surfaces together or coating a surface consisting of an acidic aqueous solution of a polyphenolic protein derived from a byssus-forming mussel.

U.S. Patent Publication No. 2005/0281775 discloses compositions based on N-alkyl acrylates for augmentation of epithelial mucosal barrier.

WO2005117844 discloses a nanocomposite drug delivery system based on chitosan and a network of colloidal nanoporous nanoparticles. WO2005056708 discloses polymers comprising a polymeric backbone and a side chain or side group containing an aromatic group substituted with one or more hydroxyl groups such as catechol for adhesion to biological surfaces to deliver a therapeutic agent.

U.S. Patent Publication No. 2005/0224175 discloses a composition consisting of an aqueous solution of a polyphenolic protein derived from byssus-forming mussel and mixing with a non-enzymatic oxidizing periodate ions.

U.S. Patent Publication No. 2007/0021569 discloses method of making sulfonated block copolymers and the various uses for such polymers. Even though it claims the usefulness of such polymers as adhesives, there are no examples to demonstrate the usefulness of such compositions as an ostomy appliance.

U.S. Patent Publication No. 2008/0085972 discloses hypoallergenic adhesive compositions based on sulfonated amphiphilic polyesters and a humectant for skin adhesion.

The prior art inventions do not demonstrate a sulfonated pressure sensitive adhesive that can support the weight of an ostomy appliance under dry and wet conditions. There is still a need for a better adhesive that attaches to skin and to wet surfaces such as mucous membrane.

### SUMMARY OF THE INVENTION

The present invention is an ostomy appliance comprising a body side component and a biocompatible pressure sensitive adhesive component for attaching the bodyside component to the peristomal area and/or stoma, wherein the biocompatible pressure sensitive adhesive component includes at least one polymer with sulfonated functional group.

### DETAILED DESCRIPTION OF THE INVENTION

According to the present invention, an ostomy appliance includes a body side component and a biocompatible pressure sensitive adhesive component for attaching the bodyside component to the peristomal area and/or stoma, wherein the biocompatible pressure sensitive adhesive component includes at least one polymer with sulfonated functional group. The sulfonation level in the polymer is sufficient to provide adhesion to wet surfaces, preferably 5-100% of the total polymer.

Further, the polymer with sulfonated functional group is selected from a group consisting of polyacrylates, polyalkylmethacrylates, N-alkylamino acrylates, N-alkylamino methacrylates, acrylamides, styrenic block copolymers, polyesters, polyvinyl ethers, polyvinyl esters, polyolefins, silicones, polyN-vinyllactams, polyurethanes, polyethers, polysaccharides, polypeptides, and combinations thereof. Furthermore, the sulfonated functional group could be in its acid, salt or amine neutralized forms, or combinations thereof.

The biocompatible pressure sensitive adhesive component could further contain other components such as hydrophobic polymers, hydrophilic polymers, amphiphilic polymers, tackifiers, resins, plasticizers, hydrocolloids, inorganic and organic particulate fillers, antioxidants, or combinations thereof.

In one embodiment, the biocompatible adhesive with the two components could be part of the appliance prior to use such as a one-piece or two-piece ostomy appliance. In another embodiment, the biocompatible adhesive could be used as a separate component to coat the stomal and peristomal area prior to attachment of the ostomy appliance. When the biocompatible pressure sensitive adhesive is a separate component, it can be delivered to the stomal and peristomal area in the form of a liquid, foam, gel, film, paste, sheet, powder, or combinations thereof.

The present invention also includes an ostomy appliance having an internal and/or external portion to the stoma and a biocompatible pressure sensitive adhesive component which includes a composition to coat a portion of the appliance for adhering said coated portion at least to the stoma and/or the peristomal area. Examples of such appliances are irrigation devices, continent ostomy port devices, etc.

The body side component of the ostomy appliance according to the present invention could further include a body waste collector component.

The biocompatible pressure sensitive adhesive of the present invention provides adhesion under dry and wet conditions.

The sulfonated group in the polymer could be randomly distributed on the repeat units or in block or alternating fashion. Some examples of sulfonated polymers are sulfonated polyesters such as those marketed by Eastman Chemical under the trade name AQ polyesters, sulfonated styrenic block copolymers, sulfonated polyolefins, sulfonated polyacrylates, sulfonated polyalkylmethacrylates, sulfonated polysiloxanes, and combinations thereof. Furthermore, the sulfonated functional group could be in its acid, salt, amine neutralized forms or combinations thereof.

The polymer with sulfonated functional group could be synthesized from sulfonated monomers such as acrylamido propane sulfonic acid, sodium styrene sulfonate, sulfonyl propyl methacrylate, etc., or by sulfonation of an existing polymer. For example, sulfonation of styrenic block copolymers is well known. Furthermore, the sulfonated polymers could be a homopolymer are copolymer. The sulfonated polymer could be linear, branched, radial, or combinations thereof. The copolymer could be block, random, branched, alternating copolymers or combinations thereof. For example, a living free-radical polymerization process could be used to synthesize a block copolymer of sulfonated unsaturated monomer such as vinyl sulfonic acid, sodium styrene sulfonate, sulfoalkyl acrylate, sulfoalky methacrylate, sulfoaryl acrylate, sulfoaryl methacrylate, with monomers such as ethyl hexyl acrylate, vinyl methyl ether, glycerol methacrylate, styrene, vinyl-polydimethyl siloxane, vinyl pyrrolidone, methyl methacrylate, etc.

### Example 1

A pressure sensitive adhesive composition may be prepared by mixing about 10-60 grams of sulfonated Styrene-ethylene-butylene-styrene (SEBS) (55-65% sulfonation on styrene, available from Sigma Aldrich) with 20-70 grams of Pentalyn H tackifier (available from Hercules Incorporated, Delaware), and 5-20 grams of naphthenic white oil such as kaydol oil (available from Sonneborn Incorporated, New York), 0-20 grams of sulfonated polystyrene (available from) and 0-10 grams of zinc stearate in a Haake twin screw mixer at about 150° C.

### Example 2

A pressure sensitive adhesive composition may be prepared by blending about 30-80 grams of a branched sulfonated polyester such as AQ1045™, 5-40 grams of a linear sulfonated polyester such as AQ55S™ (both available from Eastman Chemical Company), 0-20 grams of polyvinyl pyrrolidone PVP (available International Specialty Products), and 0-20 grams of polyethylene oxide PEG 400 (available from Dow Chemical) in a Haake mixer at 200° C. for 30 minutes.

### Example 3

A pressure sensitive adhesive composition may be prepared by blending about 30-80 grams of a branched sulfonated polyester such as AQ1045™, 5-40 grams of a linear sulfonated polyester such asAQ55S™ (both available from Eastman Chemical Company), 0-40 grams of a partially or fully hydrolyzed polyvinyl acetate, 0-20 grams of polyethylene oxide PEG 400 (available from Dow Chemical Company) in a Haake mixer at 200° C. for 30 minutes.

It will be appreciated that the foregoing description contains preferred forms of the invention, and that many modifications, improvements and equivalents are within the scope of the claimed invention.

I claim:

1. An ostomy appliance comprising a body side component, a biocompatible pressure sensitive adhesive component for attaching the said bodyside component to the peristomal area and/or stoma, wherein the biocompatible pressure sensitive adhesive component includes at least one polymer with sulfonated functional group.

2. The ostomy appliance according to claim 1, wherein the sulfonated group is 5-80% of the total polymer.

3. The ostomy appliance according to claim 1, wherein the polymer with sulfonated functional group is selected from a group consisting of polyacrylates, polyalkylmethacrylates, N-alkylamino acrylates, N-alkylamino methacrylates, acrylamides, styrenic block copolymers, polyesters, polyvinyl ethers, polyvinyl esters, polyolefins, silicones, polyN-vinyllactams, polyurethanes, polyethers, polysaccharides, polypeptides, and combinations thereof, furthermore, the sulfonated functional group could be in its acid, salt, amine neutralized forms, and/or combinations thereof.

4. The ostomy appliance according to claim 1, wherein the biocompatible pressure sensitive adhesive component could further comprise other components such as hydrophobic polymers, hydrophilic polymers, amphiphilic polymers, tackifiers, resins, plasticizers, hydrocolloids, inorganic and organic particulate fillers, antioxidants, and combinations thereof.

5. An ostomy appliance comprising a body side component, and a separate biocompatible pressure sensitive adhesive component to coat the body side component and/or the peristomal area and stoma to secure the appliance to the peristomal area and/or stoma.

6. An ostomy appliance having an internal and/or external portion to the stoma and a biocompatible pressure sensitive adhesive component includes a composition coating the portion of the appliance for adhering said coated portion at least to the stoma and/or the peristomal area.

7. An ostomy appliance comprising a body side component, a biocompatible pressure sensitive adhesive component for attaching the said bodyside component to the peristomal area and/or stoma, wherein the biocompatible pressure sensitive adhesive component includes at least one polymer with sulfonated functional group, wherein the bodyside component includes a body waste collector component.

8. The ostomy appliance according to claim 1, wherein the biocompatible pressure sensitive adhesive component attaches under dry and wet conditions.
